(19) United States
(12) Patent Application Publication
(10) Pub. No.: US 2006/0057076 A1
(43) Pub. Date: Mar. 16, 2006

(54) Oral humectants

(75) Inventors: Yasuo Hino, Osaka (JP); Shigeo Okuda, Osaka (JP); Satoshi Oshima, Osaka (JP)

Correspondence Address: BIRCH STEWART KOLASCH & BIRCH, PO BOX 747, FALLS CHURCH, VA, 22040-0747, US

(73) Assignee: YASUO HINO, suita-shi osaka (JP)

(21) Appl. No.: 10/522,248

(22) PCT Filed: Jul. 29, 2002
(86) PCT No.: PCT/JP02/07668

§ 371 Date: Jan. 25, 2005

(51) Int. Cl. A61K 31/716; A61K 8/73

(52) U.S. Cl. 424/49; 514/57

(57) ABSTRACT

The invention provides a mouth wetting agent and a mouth wetting agent for false teeth including a safe and easily usable polymer composition and capable of relieving symptoms of dry mouth by maintaining the wetting properties of an oral cavity and the stability of false teeth. The mouth wetting agent of the invention includes a polymer composition containing a pharmaceutically acceptable water-soluble polymer, a pharmaceutically acceptable polyvalent alcohol and water and/or artificial saliva.

### TECHNICAL FIELD

The present invention relates to a wetting preparation applied into an oral cavity and, more specifically, to a mouth wetting agent and a mouth wetting agent for false teeth capable of relieving symptoms of dry mouth and including a safe and easily usable polymer composition.

### BACKGROUND ART

The wetting properties of an oral cavity are maintained with saliva, but are lost upon decrease in saliva for a certain cause, thus allowing the oral cavity to be in a dry state. When the oral cavity is kept in a dry state for a long time, there occur various symptoms and functional disorders called dry mouth. The cause of such dry mouth includes various diseases causing an organic change in salivary glands, salivary gland diseases accompanying general diseases, destruction of salivary gland cells by radiation treatment, HIV infection, a reduction in secretory functions by aging, influence of various medicines administered, and mental fatigue and disorder caused by complicated social living conditions. In persons of advanced age, dry mouth is a symptom observed daily, which is considered attributable to a degenerative change in salivary glands with aging.

Insufficient secretion of saliva also adversely affects a full set of false teeth, to cause problems such as deterioration in adsorption of false teeth. Usually, a thin membrane of saliva is present between false teeth and the mucous membrane of the oral cavity, to keep the oral cavity in a negative-pressure condition thus maintaining false teeth, and also serves as lubricating oil between the oral cavity and false teeth. Even if well-fitted false teeth are used, the false teeth are poor in adsorption where the mucous membrane is in a dry state, to produce pain or bedsores in some cases upon rubbing the false teeth with the mucous membrane.

Occurrence of dry mouth causes a large number of serious pains in daily life, including not only dry feel in the oral cavity but also burning feel in the mouth, sharp pain, tongue pain, taste abnormalities, inflammation of the mucous membrane in the oral cavity, ulcer formation, clefts in the tongue and in the edge of mouth, difficulties in chewing, swallowing and speaking, and there is strong demand for suitable measures to deal therewith.

In methods of treating or relieving these symptoms at present, artificial saliva and mouthwash are used, but merely bring about transient wetting in the oral cavity. Barotin, Cephalantin and various kinds of herb medicines have been tried, but undesirable side effects are recognized and sufficient effects cannot be obtained. Thus, therapeutic methods therefor are still not established.

On one hand, there are a wide variety of commercial false-tooth stabilizers for the purpose of fixing and stabilizing false teeth, and as the commercial false-tooth stabilizers, gum-like hydrophobic synthetic polymers such as vinyl acetate resin are mainly used. They are intended for use in stabilizing false teeth for a long time in the oral cavity, and thus they are water-insoluble and cannot be expected to exhibit wetting properties, and there is worry about safety because of possible elution of a plasticizer. Highly safe naturally occurring water-soluble substances have also been studied, but they are hydrophilic polymers to be rendered heterogeneous with delayed absorption of a very small amount of water thus feeling uncomfortable upon use, and after use, their washing is troublesome due to solidification. For these disadvantages, these substances are rarely used at present.

An object of the present invention is to provide a mouth wetting agent and a mouth wetting agent for false teeth, including a safe and easily usable polymer composition and capable of relieving the symptoms of dry mouth by maintaining the wet state of the oral cavity and the stability of false teeth, to solve the disadvantages in the conventional art.

### DISCLOSURE OF THE INVENTION

As a result of intensive studies for achieving the above object, the present inventors found that a polymer composition containing a specific water-soluble polymer, a polyvalent alcohol and water and/or artificial saliva can serve as a mouth wetting agent and a mouth wetting agent for false teeth to exhibit an effect adapted to the object of the present invention; thus, the present invention has completed.

That is, the mouth wetting agent of the present invention includes a polymer composition containing a pharmaceutically acceptable water-soluble polymer, a pharmaceutically acceptable polyvalent alcohol and water and/or artificial saliva.

According to the mouth wetting agent, the wetting feel in the oral cavity can be maintained for a predetermined time, thus relieving symptoms accompanying dry mouth.

Preferable examples of the water-soluble polymer include cellulose-based polymers such as methyl cellulose, carboxymethyl cellulose, sodium carboxymethyl cellulose and hydroxyethyl cellulose, and these may be used alone or as a mixture of two or more thereof.

Preferable examples of the polyvalent alcohol include glycerin, propylene glycol and sorbitol, and these may be used alone or as a mixture of two or more thereof.

Among the above water-soluble polymers and polyvalent alcohols, a combination of sodium carboxymethyl cellulose and glycerin is particularly preferably used.

The compounding ratio of the water-soluble polymer is preferably 3 to 20 wt %, and the compounding ratio of the polyvalent alcohol is preferably 1 to 60 wt %.

By applying the above oral wetting agent onto false teeth and fitting the false teeth to the oral cavity, the false teeth can be stabilized and, simultaneously, the wetting feel in the oral cavity can be continued.

### BEST MODE FOR CARRYING OUT THE INVENTION

Hereinafter, the present invention will be described in detail.

The pharmaceutically acceptable water-soluble polymer used in carrying out the invention is not particularly limited insofar as it exhibits a thickening action upon dissolution in water, or is swollen or gelled with water to exhibit stickiness. Such water-soluble polymer includes water-soluble polymers such as naturally occurring polymers, for example, sodium alginate, gum arabic, agar, tragacanth gum, carrageenan, xanthan gum, gelatin, pullulan and dextran, semi-synthetic polymers such as dextrin, methyl cellulose, carboxymethyl cellulose, sodium carboxymethyl cellulose and hydroxyethyl cellulose, and synthetic polymers such as polyvinyl pyrrolidone, carboxyvinyl polymer, polyvinyl alcohol and Macrogol, and the like, and these may be used alone or as a mixture of two or more thereof. From the viewpoint of properties of the resulting composition, such as swelling, gelling performance, thixotropy, delayed dissolution in water and stickiness to the mucous membrane of the oral cavity, cellulose-based water-soluble polymers are preferable in the present invention, among which sodium carboxymethyl cellulose having a degree of etherification in the range of 0.7 to 1.0 is the most preferable.

The pharmaceutically acceptable polyvalent alcohol in the present invention includes glycerin, propylene glycol, sorbitol, polyethylene glycol and the like, and these can be used alone or as a mixture of two or more thereof. In the present invention, glycerin, propylene glycol and sorbitol are preferable in respect of the ability to confer flexibility on the water-soluble polymer and wetting performance in the oral cavity, among which glycerin is particularly preferable in respect of taste and safety.

The mouth wetting agent of the present invention is constituted as a polymer composition with delayed dissolution in water, comprising the water-soluble polymer and the polyvalent alcohol as essential ingredients having water added thereto to thicken, swell or gel the composition to exhibit stickiness. The amount and ratio of the ingredients incorporated into the polymer composition are generally arbitrary, but from the viewpoint of suitably keeping the handleability of the mouth wetting agent, preferably the water-soluble polymer is in the range of 3 to 25 wt % and the polyvalent alcohol is in the range of 1 to 60 wt %, more preferably the water-soluble polymer is in the range of 5 to 20 wt % and the polyvalent alcohol is in the range of 1 to 60 wt %, and still more preferably the water-soluble polymer is in the range of 8 to 15 wt % and the polyvalent alcohol is in the range of 4 to 30 wt %.

To confer suitable flexibility on the polymer composition, the polyvalent alcohol is compounded in a ratio of 50 to 200 parts by weight to 100 parts by weight of the water-soluble polymer.

As the solvent constituting the polymer composition of the present invention, water is usually used, but in respect of keeping the oral cavity in a more wet state to improve functions, a preparation having inorganic salts such as potassium chloride, sodium chloride, calcium chloride, magnesium chloride and dipotassium phosphate added to water, that is, artificial saliva is preferably used as the solvent. That is, the vapor pressure of artificial saliva is lower than that of water, and thus the polymer composition containing the artificial saliva can retain water sustainedly thus easily keeping the oral cavity in a wet state.

The mouth wetting agent of the present invention can contain not only the above constituent components as essential ingredients, but also various chemicals within a pharmaceutically acceptable range such as a preservative, a coloring agent, a flavoring and a saliva secretion accelerator, which can be provided in various forms such as sheet, tube and tablet.

When the mouth wetting agent of the present invention is used, the wetting feel in the oral cavity can be achieved by its conferred function of sustainedly releasing water molecules retained in the polymer composition. The function of sustainedly releasing water molecules can be arbitrarily regulated by changing the compounding ratio of the water-soluble polymer, the polyvalent alcohol and water and/or artificial saliva constituting the polymer composition, and the duration of the wetting feel can be established in the range from a short time (about 1 hour) to a long time (10 hours or more).

The mouth wetting agent of the present invention can be used as a novel mouth wetting agent for false teeth by applying it in a suitable amount onto false teeth and then fitting the false teeth to the oral cavity thereby achieving not only stabilization of the false teeth but also wetting feel in the oral cavity. The mouth wetting agent of the present invention, upon application to false teeth, can be used preferably as a mouth wetting agent for false teeth with improvements in total usability by suitably changing the compounding ratio of the water-soluble polymer, polyvalent alcohol and solvent constituting the mouth wetting agent thereby conferring suitable flexibility on the mouth wetting agent and improving the applicability thereof onto false teeth and the ability of false teeth to be fitted to the oral cavity.

Hereinafter, the present invention will be described in more detail by way of examples; however, the present invention is not limited to the examples.

### EXAMPLES 1 TO 5 AND COMPARATIVE EXAMPLE 1

The respective components shown in Table 1 were heated, stirred, mixed, dissolved transparently and cooled to prepare a mouth wetting agent. The compounding amounts are expressed in wt %, and the whole of the composition is 100 wt %. The artificial saliva in the compounding ingredients was prepared from the following compositions. In a comparative example, a commercially available false-tooth stabilizer made of vinyl acetate resin was used.

(Compositions of artificial saliva)

| | |
|---|---|
| Sodium chloride | 84.4 mg |
| Potassium chloride | 120.0 mg |
| Calcium chloride | 14.6 mg |
| Magnesium chloride | 5.2 mg |
| Dipotassium phosphate | 34.2 mg |
| Pure water | the balance (amount to adjust total to 100 g) |

(Evaluation of preparation as oral cavity wetting agent for false teeth)

Each of the mouth wetting agents obtained in Examples 1 to 5 and Comparative Example 1 was evaluated by a panel of 10 persons. That is, the prepared mouth wetting agent was applied onto the back of false teeth to be fitted to the oral cavity, and evaluated for its usability on the basis of the following items and evaluation criteria.

Flexibility of Mouth Wetting Agent

◯: Suitably flexible.

Δ: Slightly rigid or slightly soft.

×: Too rigid (brittle) or too soft.

Applicability onto False Teeth

◯: Easily applied.

Δ: Slightly hardly applied.

×: Hardly applied.

Fittability of False Teeth

◯: Easily fitted.

Δ: Slightly hardly fitted.

×: Hardly fitted.

Duration of Stabilization of False Teeth

⊚: Stabilization of false teeth lasts for 10 hours or more.

◯: Stabilization of false teeth lasts for several hours.

Δ: Stabilization of false teeth lasts for 1 hour or more.

×: Stabilization of false teeth is within 1 hour.

Duration of Wetting Feel in Oral Cavity

⊚: The wetting feel in the oral cavity lasts for 10 hours or more.

◯: The wetting feel in the oral cavity lasts for several hours.

Δ: The wetting feel in the oral cavity lasts for 1 hour or more.

×: The wetting feel in the oral cavity is less than 1 hour.

The results are shown in Table 1. The evaluation results are expressed on average by a panel of 10 persons.

From Table 1, it was recognized that the mouth wetting agents in Examples 1 to 5 exhibit an excellent effect on duration of the wetting feel in the oral cavity and on duration of the stability of false teeth. On the other hand, the mouth wetting agent in Comparative Example 1 was evaluated to be equivalent to the agents in Examples 1 to 5 in respect of the duration of stabilization of false teeth, but the wetting feel in the oral cavity was not recognized at all.

The mouth wetting agents used in Examples 1 and 2 had suitable flexibility, were easily applied onto false teeth, facilitated fitting of false teeth, and exhibited a lasting effect on wetting feel in the oral cavity and on stabilization of false teeth, and were thus evaluated as mouth wetting agents excellent in total usability and handleability.

### EXAMPLE 6

Whether symptoms accompanying dry mouth were relieved by applying each of the mouth wetting agents in Examples 1 to 5 onto the oral cavity of a patient with dry mouth was examined by a panel. As a result, all of the five mouth wetting agents were recognized to relieve the symptoms accompanying dry mouth, and particularly the mouth wetting agent in Example 3 had a significant relieving effect on clefts in the tongue and in the edge of mouth, and also exhibited an excellent effect that speaking was not hindered. From the above results, it is considered that a lower compounding ratio of the water-soluble polymer and a higher compounding ratio of the polyvalent alcohol are effective for use of the polymer composition of the present invention as a mouth wetting agent.

### INDUSTRIAL APPLICABILITY

For a method of treating dry mouth or relieving its symptoms, the mouth wetting agent of the present invention, unlike conventional artificial saliva or mouthwash exhibiting a transient effect, can be used as a mouth wetting agent for false teeth, based on a polymer with delayed dissolution in water and compounded with specific components, by applying it onto false teeth particularly in dry-mouth patients having false teeth put in thereby maintaining the wetting properties of the oral cavity and the stability of the false teeth to relieve the symptoms.

TABLE 1

| | | | | | | Comparative |
|---|---|---|---|---|---|---|
| Components | Example 1 | Example 2 | Example 3 | Example 4 | Example 5 | Example 1 |
| Sodium carboxymethyl cellulose | 10.0 | 15.0 | 3.0 | 25.0 | 15.0 | *Commercial |
| Conc. glycerin | 5.0 | 10.0 | 20.0 | 10.0 | 2.0 | product |
| Methyl p-oxybenzoate | 0.1 | 0.1 | 0.1 | 0.1 | 0.1 | made of vinyl |
| Pure water | Balance | — | — | — | — | acetate resin |
| Artificial salvia | — | Balance | Balance | Balance | Balance | |

Evaluation of
usability

| | | | | | | |
|---|---|---|---|---|---|---|
| Flexibility of mouth | ◯ | ◯ | Δ | Δ | Δ | Δ |
| wetting agent | | | | | | |
| Applicability of | ◯ | ◯ | ◯ | Δ | ◯ | Δ |
| false teeth | | | | | | |
| Fittability of false | ◯ | ◯ | ◯ | Δ | ◯ | Δ |
| teeth | | | | | | |
| Duration of | ◯ | ⊚ | Δ | ⊚ | ◯ | ⊚ |
| stabilization of | | | | | | |
| false teeth | | | | | | |
| Duration of wetting | ◯ | ⊚ | Δ | ◯ | Δ | X |
| feel in oral cavity | | | | | | |

1. A mouth wetting agent comprising a polymer composition containing a pharmaceutically acceptable water-soluble polymer, a pharmaceutically acceptable polyvalent alcohol and water and/or artificial saliva.

2. The mouth wetting agent according to claim 1, wherein the water-soluble polymer is at least one kind selected from cellulose-based polymers such as methyl cellulose, carboxymethyl cellulose, sodium carboxymethyl cellulose and hydroxyethyl cellulose.

3. The mouth wetting agent according to claim 1, wherein the polyvalent alcohol is at least one kind selected from the group consisting of glycerin, propylene glycol and sorbitol.

4. The mouth wetting agent according to claim 1, wherein the water-soluble polymer is sodium carboxymethyl cellulose, and the polyvalent alcohol is glycerin.

5. The mouth wetting agent according to claim 1, wherein the compounding ratio of the water-soluble polymer is in the range of 3 to 25 wt %, and the compounding ratio of the polyvalent alcohol is in the range of 1 to 60 wt %.

6. A mouth wetting agent for false teeth comprising a polymer composition containing a pharmaceutically acceptable water-soluble polymer, a pharmaceutically acceptable polyvalent alcohol and water and/or artificial saliva.

7. The mouth wetting agent for false teeth according to claim 6, wherein the water-soluble polymer is at least one kind selected from cellulose-based polymers such as methyl cellulose, carboxymethyl cellulose, sodium carboxymethyl cellulose and hydroxyethyl cellulose.

8. The mouth wetting agent for false teeth according to claim 6, wherein the polyvalent alcohol is at least one kind selected from the group consisting of glycerin, propylene glycol and sorbitol.

9. The mouth wetting agent for false teeth according to claim 6, wherein the water-soluble polymer is sodium carboxymethyl cellulose, and the polyvalent alcohol is glycerin.

10. The mouth wetting agent for false teeth according to claim 6, wherein the compounding ratio of the water-soluble polymer is in the range of 3 to 25 wt %, and the compounding ratio of the polyvalent alcohol is in the range of 1 to 60 wt %.

11. The mouth wetting agent according to claim 2, wherein the compounding ratio of the water-soluble polymer is in the range of 3 to 25 wt %, and the compounding ratio of the polyvalent alcohol is in the range of 1 to 60 wt %.

12. The mouth wetting agent according to claim 3, wherein the compounding ratio of the water-soluble polymer is in the range of 3 to 25 wt %, and the compounding ratio of the polyvalent alcohol is in the range of 1 to 60 wt %.

13. The mouth wetting agent according to claim 4, wherein the compounding ratio of the water-soluble polymer is in the range of 3 to 25 wt %, and the compounding ratio of the polyvalent alcohol is in the range of 1 to 60 wt %.

14. The mouth wetting agent for false teeth according to claim 7, wherein the compounding ratio of the water-soluble polymer is in the range of 3 to 25 wt %, and the compounding ratio of the polyvalent alcohol is in the range of I to 60 wt %.

15. The mouth wetting agent for false teeth according to claim 8, wherein the compounding ratio of the water-soluble polymer is in the range of 3 to 25 wt %, and the compounding ratio of the polyvalent alcohol is in the range of 1 to 60 wt %.

16. The mouth wetting agent for false teeth according to claim 9, wherein the compounding ratio of the water-soluble polymer is in the range of 3 to 25 wt %, and the compounding ratio of the polyvalent alcohol is in the range of 1 to 60 wt %.
